(19) United States
(12) Patent Application Publication
(10) Pub. No.: US 2026/0268481 A1
(43) Pub. Date: Sep. 10, 2026

(54) SYSTEM, METHOD AND APPARATUS TO DETECT HEALTH ABNORMALITY IN PATIENTS

(71) Applicant: Toi Labs, Inc., San Francisco, CA (US)

(72) Inventors: Vikram Kashyap, San Francisco, CA (US); Paul Cristman, San Francisco, CA (US); Deep Dhillon, Seattle, WA (US); Carsten Tusk, Des Moines, IA (US)

(73) Assignee: Toi Labs, Inc., San Francisco, CA (US)

(21) Appl. No.: 18/577,313

(22) PCT Filed: Jul. 6, 2022
(86) PCT No.: PCT/US2022/036237

§ 371 Date: Jan. 8, 2024

Related U.S. Application Data
(60) Provisional application No. 63/218,795, filed on Jul. 6, 2021.

(51) Int. Cl. G06T 7/00; A61B 5/00; G16H 50/20

(52) U.S. Cl. CPC G06T 7/0012; G16H 50/20; A61B 5/6887; G06T 2207/20081; G06T 2207/30004

(57) ABSTRACT

In one embodiment, the disclosure relates to system, method and apparatus for using Artificial Intelligence (AI) and Machine Learning (ML) to detect health abnormality using the patient's excretes using a toilette equipped with one or more sensor systems. Each sensor system may comprise hardware (e.g., detector, illuminator, etc.) and software or firmware. The sensor system may also comprise AI and ML to train the sensor to detect the user's presence, identify the user and obtain one ore more frames from the user's bathroom session. The sensor system may also obtain physical, chemical or biological data by detecting composition, color, consistence and other characteristics of the user's effluent. By using the available AI algorithm, the sensor system may then derive certain conclusions as to the health of the user based on the effluent constituents and the frequency of use.

This is a U.S. National Phase application which claims priority to the PCT Application Serial No. PCT/US2022/036237, filed Jul. 6, 2022, which itself claims priority to U.S. Provisional Patent Application Ser. No. 63/218,795, filed Jul. 6, 2021 (titled: System, method and apparatus to detect health abnormality in patients); the specifications of each application is incorporated herein in their entirety.

### FIELD

The disclosed embodiments generally relate to system, method and apparatus to detect health abnormality in patients. Specifically, the disclosure relates to system, method and apparatus for using Artificial Intelligence (AI) and Machine Learning (ML) to detect health abnormality using a patient's excretes or effluent.

### BACKGROUND

It has been recognized that visual examination of human excreta provides insight into human health. Self-reporting can be performed through diaries, but is subject to interpretation and bias. The ability to objectively and consistently assess excreta on a regular basis can help track symptoms of dysfunction and determine the effects of medications, diet, lifestyle, supplements, and other interventions.

The major components of the American bathroom, including the toilet and mirror, have not fundamentally changed for more than 100 years. In the United States, the toilet mostly lacks any electrical, sensor or network connected capabilities. In Japan, the toilet seat is widely used as an electric bidet for reasons related to hygiene. The vast majority of bathroom mirrors across the world lack any electrical, sensor or network connected capabilities, especially related to health or wellness.

There is a need for accurate, convenient and unbiased electronic biomonitoring capabilities that analyze excreta and other health-related characteristics in a bathroom setting.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates components of an exemplary system according to one embodiment of the disclosure;

FIG. 1B illustrates a sensor positioned according to one embodiment of the disclosure;

FIG. 1C shows the flow-diagram for an exemplary software architecture according to one embodiment of the disclosure;

FIG. 2 illustrates an exemplary male/female ratio for each participating community;

FIG. 3 illustrates an exemplary annotation timeline for an exemplary study;

FIG. 4 illustrates an exemplary convolutional neural network (CNN) architecture according to one embodiment of the disclosure;

FIG. 5 demonstrates a comparison between percent of entries logged by TrueLoo™ Sensor (the “TL Sensor”) and the operator on an hourly basis;

FIG. 6 demonstrates a per-resident comparison of bowel movement entries by the TL Sensor versus the operator entries;

FIG. 7 shows the number of urinations missed per resident in the month of November;

FIG. 8 shows missing log entries by each toileting session type;

FIG. 9 demonstrates the percentage of missing stool session log entries by operator shift time and month;

FIG. 10 demonstrates percentage of missing urine session by log entries by shift time and month for ALR #2;

FIG. 11 demonstrates the distribution in stool size and the variability in their descriptors;

FIG. 12 demonstrates the variability in stool consistency distribution descriptors;

FIG. 13 demonstrates the percentage of missed stool sessions by participants;

FIG. 14 demonstrates the number of daily BM classified by operators versus the actual events detected by the sensor;

FIG. 15 demonstrates the relative formed and unformed stool frequency capture;

FIG. 16 demonstrates the formed stook frequency and presence of frank blood captured by the TL Sensor; and

FIG. 17 demonstrates total urination and cloudy urination frequency captured by TrueLoo.

### DETAILED DESCRIPTION

In the following description, numerous specific details are set forth in order to provide a thorough understanding of various embodiments. However, various embodiments may be practiced without the specific details. In other instances, well known methods, procedures, components, and circuits have not been described in detail so as not to obscure the particular embodiments. Further, various aspects of embodiments may be performed using various means, such as integrated semiconductor circuits (“hardware”), computer-readable instructions organized into one or more programs (“software”), or some combination of hardware and software. For the purposes of this disclosure reference to “logic” shall mean either hardware, software, firmware, or some combination thereof.

References to “one embodiment,” “an embodiment”, “example embodiment”, “various embodiments”, etc., indicate that the embodiment(s) of the invention so described may include particular features, structures, or characteristics, but not every embodiment necessarily includes the particular features, structures, or characteristics. Further, some embodiments may have some, all, or none of the features described for other embodiments.

In the following description and claims, the terms “coupled” and “connected,” along with their derivatives, may be used. It should be understood that these terms are not intended as synonyms for each other. Rather, in particular embodiments, “connected” is used to indicate that two or more elements are in direct physical or electrical contact with each other. “Coupled” is used to indicate that two or more elements co-operate or interact with each other, but they may or may not have intervening physical or electrical components between them.

As used in the claims, unless otherwise specified the use of the ordinal adjectives “first”, “second”, “third”, etc., to describe a common element, merely indicate that different instances of like elements are being referred to, and are not intended to imply that the elements so described must be in a given sequence, either temporally, spatially, in ranking, or in any other manner.

Discussions herein utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Various labels may be used in describing particular devices, software, functions, etc. These are used for simplicity and convenience, but this should not be interpreted to mean that only items with those labels are covered by the description. Devices, software, functions, etc., that perform in the same manner as the described items, but are labeled with different terminology, should be considered to be equivalent to the described items.

Various embodiments of the invention may be implemented fully or partially in software and/or firmware. This software and/or firmware may take the form of instructions contained in or on a non-transitory computer-readable storage medium. Those instructions may then be read and executed by one or more processors to enable performance of the operations described herein. The instructions may be in any suitable form, such as but not limited to source code, compiled code, interpreted code, executable code, static code, dynamic code, and the like. Such a computer-readable medium may include any tangible non-transitory medium for storing information in a form readable by one or more computers, such as but not limited to read only memory (ROM); random access memory (RAM); magnetic disk storage media; optical storage media; a flash memory, etc.

The term “wireless” may be used to describe circuits, devices, systems, methods, techniques, communications channels, etc., that communicate data by using modulated electromagnetic radiation through a non-solid medium. A wireless device may comprise at least one antenna, at least one radio, at least one memory, and at least one processor, where the radio(s) transmits signals through the antenna that represent data and receives signals through the antenna that represent data, while the processor(s) may process the data to be transmitted and the data that has been received. The processor(s) may also process other data which is neither transmitted nor received.

As used within this document, the term “communicate” is intended to include transmitting and/or receiving. This may be particularly useful in claims when describing the organization of data that is being transmitted by one device and received by another, but only the functionality of one of those devices is required to infringe the claim. Similarly, the exchange of data between a network controller and a mobile device (both devices transmit and receive during the exchange) may be described as ‘communicating’, when only the functionality of one of those devices is being claimed.

An exemplary embodiment that is described throughout the disclosure is the so-called 143 TrueLoo™ model (interchangeably, the TrueLoo™ or the “TL Sensor”). The reference to this model is exemplary and in no way limiting of the disclosed principles.

An exemplary TL Sensor System may include hardware, software or a combination of hardware and software (i.e., firmware). The hardware component may comprise a physical toilette and sensor components. The sensor components, as described further below, my be positioned in/on the toilette to allow detection of user's effluent. The sensor component may also comprise means for detecting one or more of physical, chemical or biological components of the user's effluent. The detected components may then be communicated and processed through an Artificial Intelligence (AI) system which may be trained to detect and identify (e.g., with Machine Learning (ML) algorithms) to identify constituents or user behavior by comparing the obtained data with existing models. In one embodiment, the sensor system may additionally capture images of the user's effluent sample and compare those with existing data to arrive at a conclusion (i.e., diagnosis) regarding the user's health. These exemplary embodiments are further illustrated in relation to the experimental observations provided in the various studies discussed herein.

Several TL Sensors were deployed in a clinical study over a period of 12+ months. Data captured by each TL Sensor was centralized and used to create a “gold-standard” dataset that was further validated and reviewed by Board-Certified Gastroenterologist Subject Matter Experts (SMEs). This dataset involved creating a series of labels reflecting “digital biomarkers” (DBMs) for excreta and toileting event imagery. By assigning DBMs to images collected by the TL Sensor, machine learning (ML) models have been trained to extract relevant information on stool consistency, stool color, stool frequency, stool size, urine color, urine clarity, urine duration, and urination frequency. To date, more than 2 million images have been captured by the TL Sensor. Labels have been assigned to 40,000 images, which translates to 10,000 toileting sessions, and over 100,000 DBMs.

Stool and urine data collected by 10 caregivers at a high-end assisted living community was compared to electronic logs generated by the TL Sensor. The exemplary TL Sensor recorded almost 50% more bowel movements (BMs) than staff, and staff only recorded 10% of the urinations captured by the TL Sensor. These results were consistent when also looking at 3 residents in a boutique dementia care facility. The rate at which operators miss bowel movements and urination events demonstrates the limitations of caregivers in performing this task. This includes not documenting important excreta characteristics such as cloudy urine, loose stools, and suspected bleeding—all of which the TL Sensor reliably captured. The TL Sensor's electronic monitoring of stool and urine characteristic changes were also associated with conditions including urinary tract infections, dehydration, Clostridium difficile infection, microscopic colitis, kidney stones and antibiotic use.

The TL Sensor was well-accepted by the users, with more than 90% reporting that the system required zero effort to use. The majority of those surveyed stated they would prefer to have the TL Sensor monitor bowel and bladder issues rather than have to bring it up proactively to their caregivers.

Monitoring basic activities of daily living, (ADLs) serves as an important predictor of the need for alternative living arrangements (i.e., higher acuity care), especially in senior-living. In a healthcare setting, assessing ADLs is crucial in helping providers and caregivers assess the patient's health condition, treatment plan, and intervene appropriately. ADLs are used to manage one's basic physical needs, which includes toileting. Besides common bladder issues such as urinary tract infections, bowel problems such as constipation, diarrhea and fecal incontinence are highly prevalent among senior-living residents, and present several challenges for care staff. Nurses tend to spend a significant amount of time managing residents' bowel problems; however, there is limited evidence on the consistency, accuracy and objectivity of this manual, labor-intensive process. Toi Labs, Inc. has created the TL Sensor, a connected toilet seat that is designed to automatically log and monitor toileting sessions, and notify users of any changes in stool and urine. Reporting and monitoring stool and urine characteristics has shown to contribute to notable improvements in the quality of care that residents in senior-living facilities receive, especially those living with several comorbidities. The exemplary TL Sensor aims to make this reporting more reliable, providing caregivers with data to improve their clinical decision through evidence-based technology.

The disclosed embodiments demonstrate the feasibility of the TL Sensor to log meaningful clinical changes in excreta in an older adult population. This involved collecting a large amount of data to understand if long term monitoring of changes in stool and urine are clinically significant, or if non-clinical variables, such as diet, could make it especially challenging to identify these changes. The presented retrospective examples and study aim to highlight the value of accurately monitoring stool and urine changes, and the feasibility of the TL Sensor to impact the slowing down of disease progression, or monitoring prescribed treatments.

### Study Objectives

The primary objective and endpoint of this study was to create an automated and personalized urine and stool dataset based on the TL Sensor image analysis. To achieve this, a large volume of images and toileting sessions were labeled by human annotators in order to train a machine learning system to automatically process the data.

The secondary objectives of this study were as follows: First, to compare the created the TL Sensor dataset to the current methodologies; specifically, caregiver-reported and self-reported methods of bowel movement and urinary event tracking. Second, to determine the clinical relevance of stool and urine characteristics by identifying and comparing bowel movement and urinary event data collected by TrueLoo™ to data collected through health history (i.e., medical records) and urine sampling.

### Methods

Study Design—The study was intended to be carried out over the course of 8 months but was extended due to the COVID-19 pandemic, and to increase the amount of data collected. There was only one group, or arm, of subjects; in other words, all subjects belonged to the same study group, regardless of symptoms, disease diagnosis or state. Furthermore, there was no stratification of the population by sex, age, race or disease severity. Each subject was monitored in their residences within the community. The study was observational and retrospective, meaning all data could be analyzed after it was collected with no change in care so having only one study group is reasonable. The study had some practical differences based on the level of care the participants received. This resulted in two groups of participants, independent living residents (“ILR”) and those in assisted living (“ALR”) or memory care settings (also grouped as ALR). The key difference in the two groups was how the data was collected for the monthly assessments. For ILR, the monthly assessments were done with the resident relying on their own self reporting. For ALR, the monthly assessments were done in coordination with the staff providing assistance to the residents and allowed comparison to caregiver reporting of bowel movements and urinations.

After each participating resident completed the informed consent process, a toilet equipped with the TL Sensor was installed in their private bathroom along with an initial health assessment to collect demographic information and any pre-existing conditions. After these initial activities were completed, on a monthly basis a short assessment check in was performed to document any changes in condition. The monthly assessment served as the basis to compare what the residents were able to self-report in comparison to what the TL Sensor recorded.

As the study was designed to have the final analysis done after the data was collected, retrospectively, health records were only released at the end of a resident's participation. In some cases, where it was known from self or staff reporting that a resident had a serious clinical episode such as a hospitalization, the confirmatory medical records were sought before the end of the study.

At the end of the study, a survey about user satisfaction was given and results collected. This survey can be seen in Appendix B.

### Description of Exemplary Technology

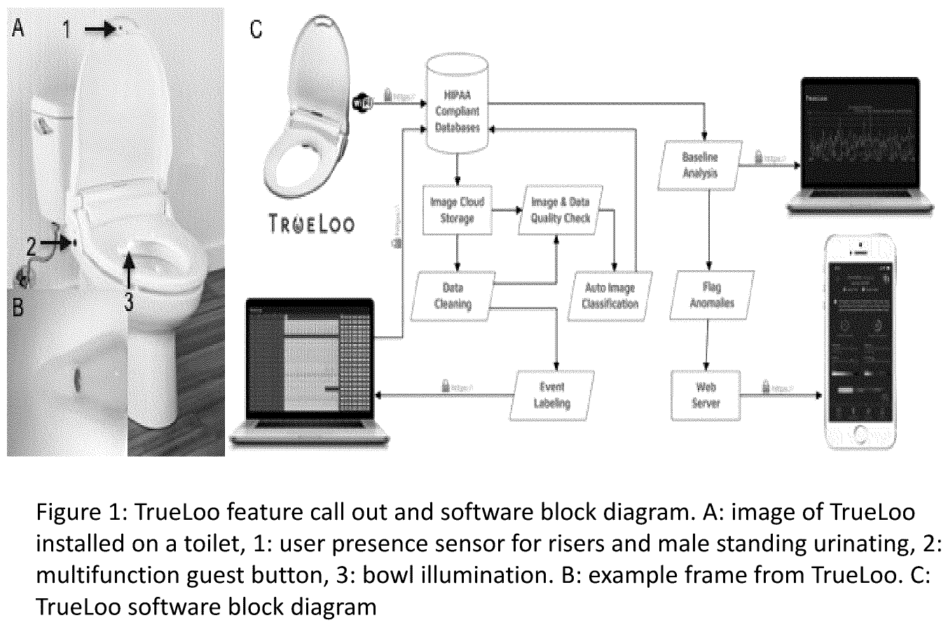

An exemplary TL Sensor system consists of two parts: a hardware component which is delivered as a replacement toilet seat, and a software system for analysis and reporting. FIG. 1A is a photograph of an exemplary toilette and sensor with features called out and an image captured from the optical system. The TL Sensor seat has two user presence sensors: one contact sensor bounded to the seat with no visible sign to the user and a second non-contact time of flight distance sensor (FIG. 1A.1) to activate when the user does not sit on the seat, such as a male standing urinating. By using the two sensors the system can distinguish between standing and seated events as well as non-event classification. The rear housing is used to mount the optical system and support electronics. The bowl is illuminated (FIG. 1A.3) uniformly by RGBW LEDs to control color balance and some narrow band imaging illuminating with only one color. This allows for consistent imaging conditions for all currently encountered toilet geometries. Not shown is the RGB 8 Megapixel manual focused camera and needed control and communication electronics. The system is powered by a single board computer with integrated WiFi communications for transmitting the images. The TL Sensor seat has a guest button (callout B) to disable the system if a guest needs to use the toilet. The guest button is required as part of the current clinical studies and automatically resets after pressed. In an exemplary implementation, no image was recorded from guest events but they are registered in the database as an activation of the TL Sensor. Not shown in this figure is the cable routing using conduit to fix the cable to the wall and connect the unit to power using a wall mount type AC/DC transformer at the outlet allowing for a long cable run with a low voltage thin wire and no need for a new outlet or needing to replace or recharge batteries. The seat is fixed to the toilet using a standard commercial mounting system for replacement toilet seats. After the TL Sensor seat is installed, it should require minimal to no ongoing maintenance other than ensuring that the optics stay clean.

When a user activates the TL Sensor by sitting on the seat or standing in front of the toilet the system will activate an event and immediately start imaging. It should be noted this is a full resolution image. Full resolution video may be used without departing from the disclosed principals. The TL Sensor continuously captures images (frames for video use) for the duration of time the user is seated or standing in front of the device. Immediately after the event is finished the images are transferred over WiFi to Toi Labs HIPAA compliant servers for storage and analysis.

### Inclusion Criteria

To participate in the study, each participant must have satisfied all of the following inclusion criteria: (i) Must have been willing to participate and provide consent for the study; (ii) Must have been a male or female, aged 55 or older; (iii) Must have been a resident of a senior living facility; (iv) Must have had regular access to a TL Sensor.

### Exclusion Criteria

Participants that satisfied any of the following exclusion criteria were not be permitted to enroll in the study: (i) Candidates who were unwilling or unable to accept the requirements associated with installing the TL Sensor in their residence, including power and WiFi connectivity; (ii) Candidates who used certain types of toileting assistance devices that at this time are not compatible with the TL Sensor (e.g., padded toilet seat risers).

### Study Demographics

One hundred and forty three TL Sensor units were deployed in a clinical study over a period of over twelve months. Of those deployed, 98 participants were active and involved with monthly phone check-ins with a registered nurse that assessed each participant's health, including medication changes and recent problems that they may have experienced with their stool or urine. Also, noted is that 8 of the 90 TL Sensor were shared by cohabiting couples. This did not result in exclusion but both residents needed to participate in the study and consent process.

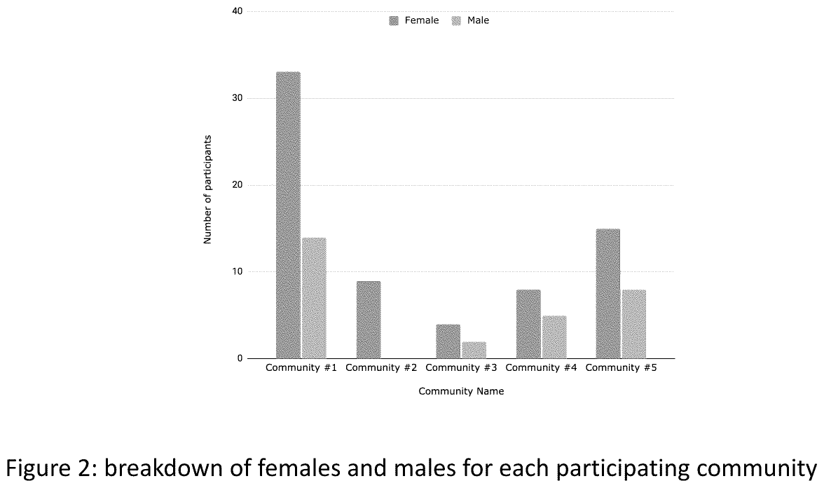

The participants in the study were between 67 and 99 years old, with the average age being 86 years old. Study participants were spread out across five high-end senior living communities across Northern California, three of which were categorized as independent living (i.e., Community #1, #4, and #5), while two were non-independent (i.e., Community #2 and #3), and included residents in assisted living and memory care. About 47.95% of study participants resided in Community #1, while 9.18% were from Community #2, 13.26% from Community #4, 6.12% from Community #3, and 23.47% from Community #5 residences. The majority of study participants were female, with a breakdown of 70.4% female and 29.59% male. FIG. 2 shows the breakdown of females and males for each participating community.

The primary reason for discontinuation of the study and premature withdrawal was due to an administrative problem that occurred with one senior living operator. The administrative problem was the sudden loss of support from senior management of the senior living operator that blocked us from contacting the staff in their communities. Therefore, all of the ALR were withdrawn because the monthly collection of updated health information from the staff could not occur. The study did continue with the operator's ILR, where we could still directly engage with participating residents. This accounted for 31 of the 45 residents who withdrew from the study. The remaining participants who withdrew were due to a variety of reasons including changing their minds, moving out of the community, and unfortunately, death.

### Results

Data collection—The study was successful in generating a large volume of data with over 60 million individual images collected, and over 350,000 activations or toileting sessions. A total of 775 monthly check-in assessments were performed. To date, a total of 30 medical records have been released as part of the study.

Data monitoring—Taking advantage of the non-battery power and WiFi connectivity configuration, the TL Sensor data was logged and monitored to ensure the device was connected and working properly. In the event a TL Sensor went offline, the gap was recorded as such and the resident or community was contacted to reset the unit or reconnect it to power, depending on the situation. In some cases, the WiFi network was the problem and network troubleshooting was performed. The exemplary TL Sensor saved approximately 1 week of data locally and resumed uploading data when a WiFi connection is reestablished. This capability provides added capture/integrity in the event of a prolonged WiFi outage. However, if the TL Sensor was completely unplugged there was the possibility of missing data. The gaps in data that occurred had no significant impact on the study or findings.

The monthly assessments were logged and checked against the master participant list to ensure all residents were contacted. Some monthly assessments were missed when the resident was unavailable (e.g., on vacation and not using the TL Sensor).

The final retrospective review of the medical record and the TL Sensor data augments the monitoring of the TL Sensor data and collection of monthly assessments.

### Data Management

The management of the data was done differently depending on the data source or type. The TL Sensor data was managed using Amazon Web Services (AWS) HIPAA compliant databases and image storage. The assessment data was stored using HIPAA compliant Forms with limited access. Similarly, the medical records were digitized and stored in a secure HIPAA compliant Drive.

Through each monthly data review of the TL Sensor data, sessions of interest that were captured by the system were directly compared to self-reported information from each resident. To determine the discrepancies in sessions that were observed by the TL Sensor and reported, observed by the TL Sensor and not reported, or not observed by the TL Sensor and reported, all of the data was aggregated into a spreadsheet summarizing findings gathered by the TL Sensor and those determined by the participant's self-reporting. Notable sessions that occurred on a monthly basis were described as monthly adverse episodes.

The number of monthly adverse episodes, where more specifically, monthly adverse episodes are a collection of adverse episodes that have occurred during a given month, was determined through the spreadsheet which tallied these occurrences. The total number of monthly adverse episodes picked up by the TL Sensor was 736 adverse episodes. The breakdown of adverse episodes, by type, is as follows:

TABLE 1
Breakdown of adverse episodes by type

| Type | # of adverse episodes |
|---|---|
| Constipation | 196 |
| Diarrhea | 364 |
| Cloudy urine | 91 |
| Bleeding | 85 |

Sessions were analyzed and categorized into different types of adverse episodes. To standardize this categorization, specific definitions of each adverse episode were created. In order for a series of sessions to be counted as a monthly adverse episode, one of the following requirements must've been met:

Constipation: data captured by the TL Sensor demonstrating that no stool events have occurred during a consecutive 72-hour period.

Diarrhea: data captured by the TL Sensor demonstrates a noticeable stool stream, is completely liquid, or otherwise contributes to diarrhea (i.e., unformed stool) for a consecutive 24-hour period.

Cloudy urine: data captured by the TL Sensor demonstrates cloudy urination (i.e., events clearly showing cloud formation during urine dispersal in toilet water) and diagnosed UTIs (i.e., diagnoses that were directly reported by the resident).

Bleeding: data captured by the TL Sensor demonstrates the potential presence of frank blood (dark red liquid), or melena (i.e., black tarry stool). This includes blood on toilet paper.

### Machine Learning

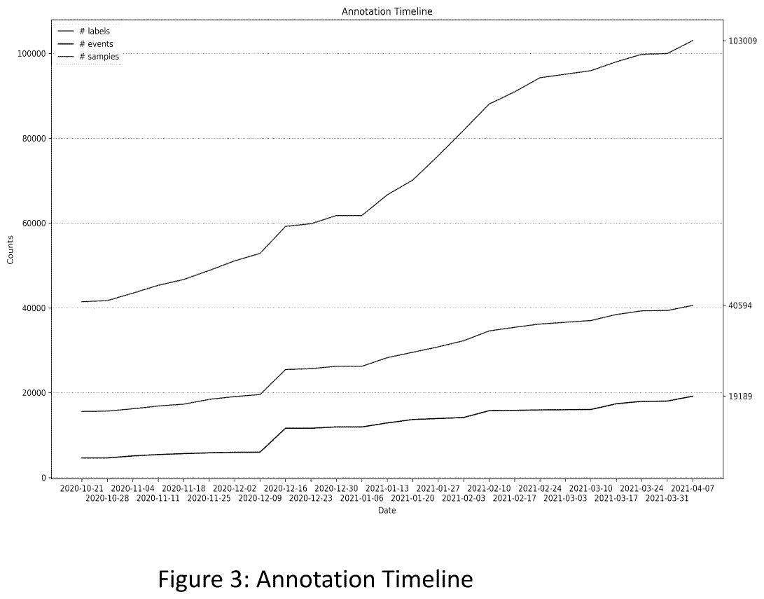

A panel of Board-Certified Gastroenterologist Subject Matter Experts (SMEs) was enlisted to create a “gold-standard” database. The SMEs created a rule set for image annotators, who are lay people trained to accurately identify image content. These image annotators labeled part of a dataset containing more than 2 million images. These labeled images were then used to train the Machine Learning (ML) algorithms before being run on the full dataset. The applied labels were used to create “digital biomarkers” (DBMs) for excreta and toileting event imagery. To date, the annotators have labeled more than 10,000 sessions (times people have used the toilet) with more than 40,000 images and over 100,000 DBMs. FIG. 3 shows the progress in annotations over the last 6 months of labeling.

An exemplary list of commonly used labels includes separate_hard_lumps_nut_like_stool; lumpy_sausage_like_stool; smooth_sausage_snake_stool; sausage_like_with_surface_cracks_on_stool; fluffy_mushy_stool_ragged_edges; soft_blobs_distinct_edges_stool; watery_liquid_no_solids_stool; unformed_stool; formed_stool; small_quantity_bright_red_blood_present_stool; large_quantity_bright_red_blood_present_stool; bright_red_blood_present_stool; dark_red_black_blood_present_stool; toilet_paper_present; menstrual_products_present; pale_yellow_urine_color; dark_yellow_urine_color; clear_urine_color; cloudy_urine; orange_urine_color; red_urine_color; urine_stream_present; urination_underway; urine_present; stool_present; blood_present; vomit_present.

To be effective, in an exemplary embodiment, TrueLoo™ may be able to obtain session level results, not merely frame level accuracy. To accomplish this, many labels are examined over time progression. For example, if a session starts with an image of a “clean bowl”, the rest of the images are classified with that as the starting point. If, however, the session started with stool already in the toilet from a previous unflushed event, the frames are processed in a different way. Each individual label does not have to be 100% accurate to be able to obtain highly accurate session results. At available accuracy levels, the correct identification of labels is robust enough for session-labeling using multiple images within a session.

A toilet session can have hundreds of images, and each image has many applied labels. The DBMs are defined for the entire toilet session, extracting the relevant information on stool consistency, stool color, stool frequency, urine color, urine clarity, urine duration, and urination frequency. In order to build the algorithms, we established a rule set for the ML to follow, e.g., if more than 10 DBMs on different images intra-session are labeled as watery liquid stool, the session is classified as an unformed stool session. It would be classified in this fashion even if there were 30 images associated with the session, and only 10 DBMs tag diarrheas. In this way, we are able to properly categorize full toileting sessions even if the ML is not fully accurate for each label created. It is worth mentioning that the rule sets themselves are also learned from the data by labeling on the session level and using the frame level result labels as input.

Discussion—ML Development for Creation of Complete Stool and Urine Dataset with Manual Review (Endpoint #1):

Once the images are collected by the TL Sensor they were processed to create a complete dataset for the individual. To effectively review the volume of images created a number of steps are taken. First is a ML algorithm to analyze every frame of a toileting session. The study succeeded in developing the frame level algorithm over the last 6 months that can identify over 25 DBMs. See Table 2 for current precision, recall, and F1 scores of select DBMs in the base model. The base model represents a multi-label convolutional neural network.

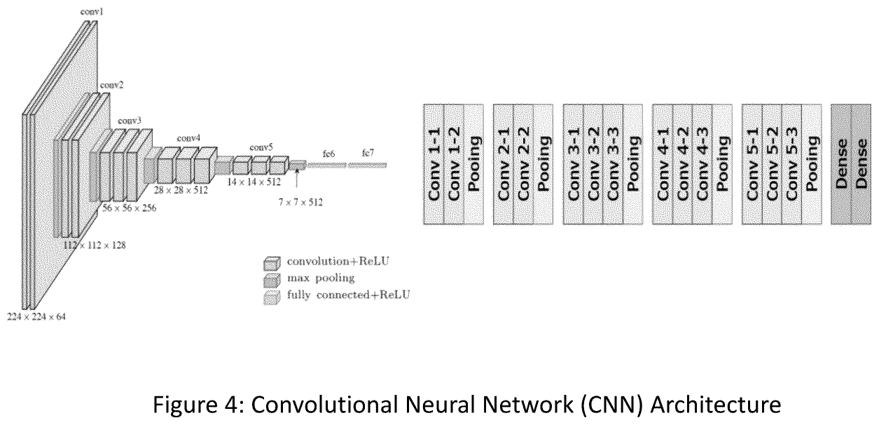

A classic deep learning network structure was used for the initial neural network architecture, as illustrated in FIG. 4. The network consisted of five convolutional blocks with max-pooling for feature learning and extraction followed by two dense layers. A sigmoid activation function on the final layer was used for the final multi-label classification task and a binary cross entropy loss function. The reasoning behind this choice is that this particular architecture has been very successful in traditional image classification tasks and pre-trained weight configurations based on classic benchmarks are readily available, making it an excellent candidate for fine-tuning and transfer learning. Other more refined architectures can be used without departing from the disclosed principles.

TABLE 2
for current precision, recall, and F1 scores of select Frame Labels

| Frame Label | Precision | Recall | F1-score |
|---|---|---|---|
| Blood Present | 0.887 | 0.738 | 0.805 |
| Urine Present | 0.877 | 0.921 | 0.898 |
| Urination Underway | 0.795 | 0.742 | 0.768 |
| Formed Stool | 0.877 | 0.89 | 0.883 |
| Unformed Stool | 0.886 | 0.896 | 0.891 |
| Separate Hard Lumps Nut Like Stool | 0.554 | 0.557 | 0.556 |
| Lumpy Sausage Like Stool | 0.674 | 0.457 | 0.544 |
| Sausage Like With Surface Cracks On | 0.622 | 0.625 | 0.624 |
| Stool | | | |
| Smooth Sausage Snake Stool | 0.578 | 0.472 | 0.52 |
| Soft Blobs Distinct Edges Stool | 0.551 | 0.428 | 0.482 |
| Fluffy Mushy Stool Ragged Edges | 0.72 | 0.695 | 0.707 |
| Watery Liquid No Solids Stool | 0.749 | 0.725 | 0.737 |
| Toilet Paper Present | 0.846 | 0.861 | 0.853 |
| Bodily Appendage | 0.938 | 0.855 | 0.895 |
| Clean Bowl | 0.803 | 0.707 | 0.752 |

The session ML model has recently been developed from the collected data. Currently only the most basic classification of stool or urination is reliable in the ML model. The reason is of the approximately 13,000 labeled sessions used to train the model approximately 7,000 are urinations and 3,000 stool sessions with flush sessions the next most common with 1,000 sessions and remaining sessions are in the low 100s. This limited supply of samples showing the remaining session types presents challenges, as approximately 1,000 sessions are needed for stable model performance and inclusion. The latest precision, recall, and F1 scores for the sessions model are shown in table 3 for stool and urination sessions only. The labeling of sessions is ongoing and it is expected other labels will soon cross the 1,000 label threshold for inclusion.

TABLE 3
Current precision recall and F1-score
for urination and stool sessions

| Session Label | Precision | Recall | F1-score |
|---|---|---|---|
| Urination session | 0.968 | 0.978 | 0.973 |
| Stool session | 0.969 | 0.953 | 0.961 |

In one embodiment, the TL Sensor system uses manual human identification of changes in toileting patterns with the aid of the frame level model. The development of the episode model is beyond the scope of the current study. In one implementation, the TL Sensor system with human support was able to prove the feasibility of identifying clinically relevant changes in data.

### Comparisons to Self-Reporting and Staff Logs to Identify Anomalous Toileting Patterns (Endpoint #2)

The TL Sensor data and nurse assessment analysis: ILRs—Independent Living Residents involved in the study underwent monthly assessments conducted through a phone call by a Registered Nurse (RN). The purpose of the assessments was to benchmark the data captured by the TL Sensor with relevant sessions that were self-reported by participants. This highlighted the unreliability of residents in recounting their stool and urine habits, or even detecting any noticeable changes that could otherwise be clinically significant. Through the 775 assessments that were conducted across t ILRs, the results were as follows:

TABLE 4
Breakdown of monthly nurse assessments comparing
observations by the TL Sensor to self-reporting

| | observed and | observed but not | not observed but |
|---|---|---|---|
| episode type | reported | reported | self-reported |
| constipation | 55 | 80 | 34 |
| diarrhea | 99 | 202 | 24 |
| cloudy urine | 13 | 48 | 10 |
| blood | 14 | 55 | 1 |

The questionnaire carried out by the RN can be seen in Appendix A. As seen in the table above, 355 of adverse episodes were observed by the TL Sensor, but were not self-reported by the resident. This shows that, for example, 51.8% of adverse episodes go unreported by residents. It is important to note that adverse episodes that were not observed by the TL Sensor but were self-reported are likely explained by recall bias, or discrepancies in the resident's understanding of diarrhea, constipation, etc. and the clinical description of these episodes that our study had established.

The TL Sensor data comparison to operator data: ALRs—Toilet logging is especially prevalent in assisted-living communities. Data collected by human operators at three assisted-living communities was compared to the TL Sensor data collected from the same set of residents. For the first analysis involving ALR #1, stool and urine logging data for 9 residents was collected by 10 caregivers. For the second analysis involving ALR #2, stool and urine logging data for 3 residents was collected. The third analysis involving ALR #3, stool and urine data for a single resident was collected and compared to data picked up by the TL Sensor. The communities maintained different logging standards or types. This is common as there is no standardization in toilet logging. In two cases (i.e., ALR #1 and #3) the staff entered the information into an electronic record system that logged the user and time of entry. Logs for ALR #2 were entered by shift in a database. In all cases, comparing data captured by the TL Sensor with toilet logging collected by human operators demonstrated that the TL Sensor was more accurate and consistent in logging toileting sessions.

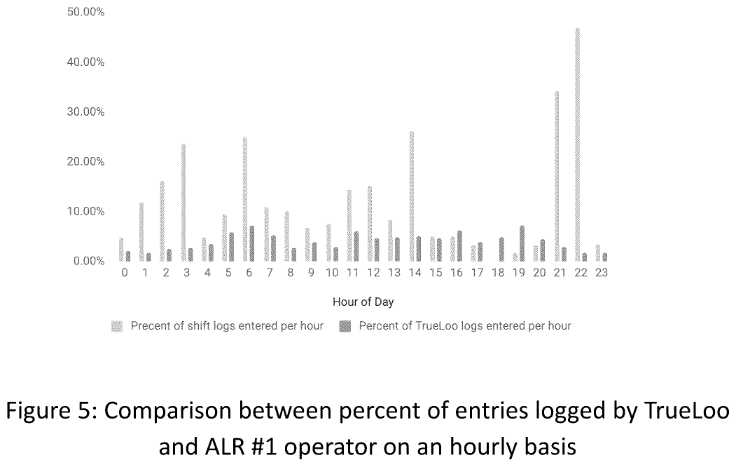

Analysis of ALR #1—The disclosed TL Sensor demonstrated an ability to log toileting sessions in a timely manner, especially when benchmarked against operators of ALR #1. On average, over the course of 24 hours, the TL Sensor system logging exhibits a normal distribution, as seen in FIG. 5, while operators from ALR #1 tend to log toileting sessions at shift changes (i.e., 6 AM, 2 PM and 10 PM). This delay in logging can contribute to errors in recording crucial details (such as stool consistency, size, color, or urine clarity and color) which can be significant factors in assessing a resident's condition, disease progression, or the effectiveness of a prescribed treatment.

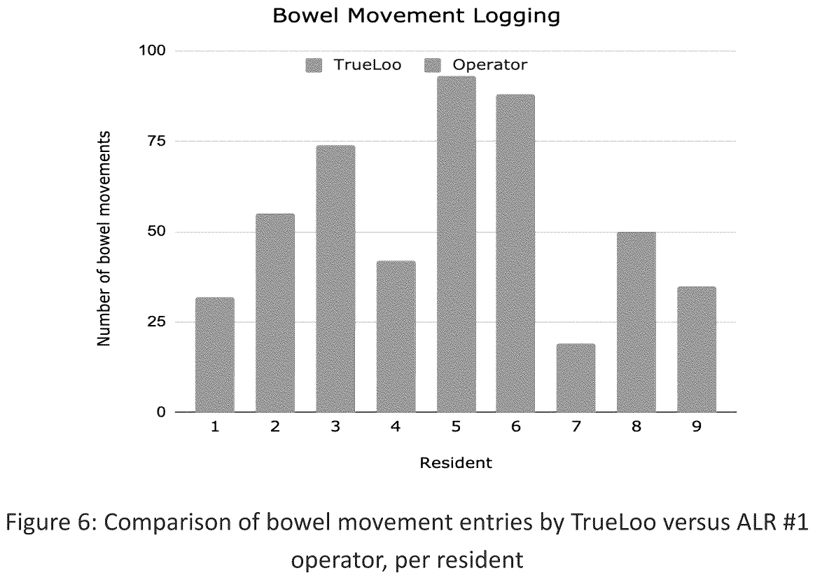

Operator logs for the month of November 2020 were further compared to all data captured by the TL Sensor for the same time period. Results showed that 43% of stool events that were picked up by the TL Sensor were missed by ALR #1 staff. FIG. 6 below shows the number of bowel movements missed per resident in the month of November.

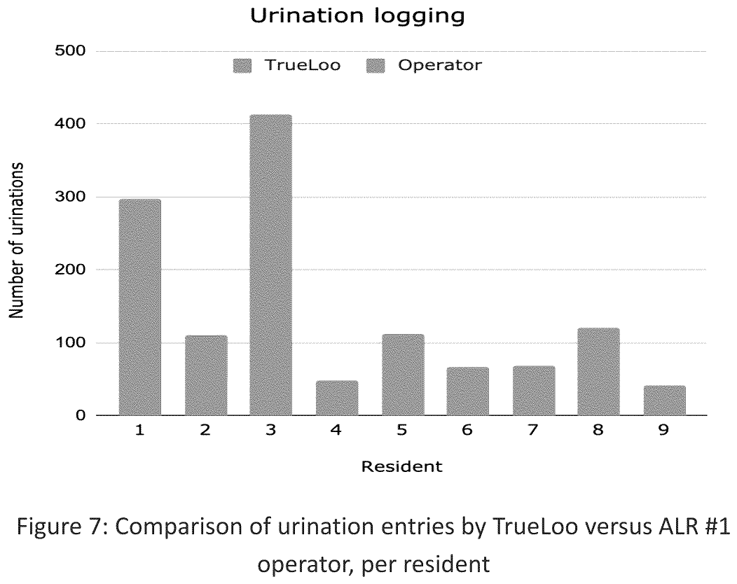

Furthermore, operators from ALR #1 missed 82% of urination events that were captured by the TL Sensor. FIG. 7 shows the number of urinations missed per resident in the month of November.

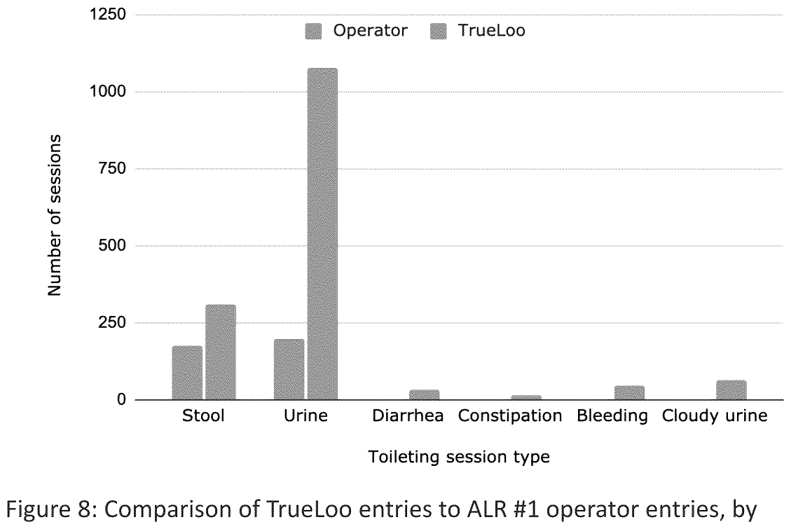

Operators from ALR #1 additionally failed to report in the log adverse episodes captured by the TL Sensor, including details of stool consistency, the presence of blood, or indications of cloudy urine. While the TL Sensor identified that there were 32 instances of unformed stool, 48 of bleeding, 14 of constipation, and 63 of cloudy urine sessions, operators failed to capture these adverse episodes. It is possible the staff logged some of this information in other parts of the resident's record not part of the transferred data. FIG. 8 shows missing log entries by each toileting session type.

Although the ALR #1 operator log format requires care staff to document 3 features for residents' bowel movements and urinations, only 7% of bowel movement logs and 39% of urine logs were completed. The incomplete records were observed more often than the ones updated by the night shift (50% of incomplete records are contributed by the night shift, and 22.5% are from the morning, and 27% from the afternoon shift). Lastly, although 10 caregivers were expected to assist with logging activities, more than 50% of logged sessions were done by two caregivers, and did not include log entries outlining the required details of stool (i.e. size and consistency) and urine (i.e. clarity) sessions.

Analysis of ALR #2—Data collected from 3 residents by operators from ALR #2 was compared to the TL Sensor's findings over the course of five months (i.e. from August 2020 to December 2020). Note that initially, the TL Sensor data was collected from five residents from ALR #2, however, two of them were wheelchair users. These users rarely use the toilet directly and their data was solely reliant on care staff dumping excreta into the toilet. The TL Sensor captured less stool sessions overall due to the lack of overall usage. As there exist many uncontrolled variables in collecting data from wheelchair users, these individuals were excluded from the following analysis.

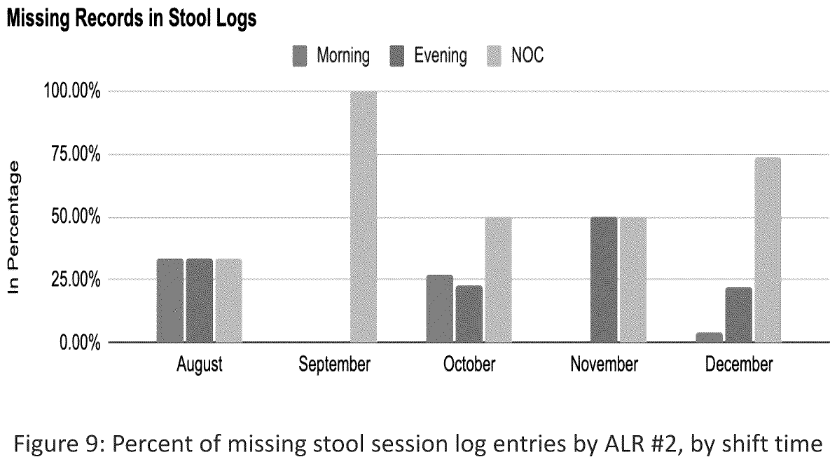

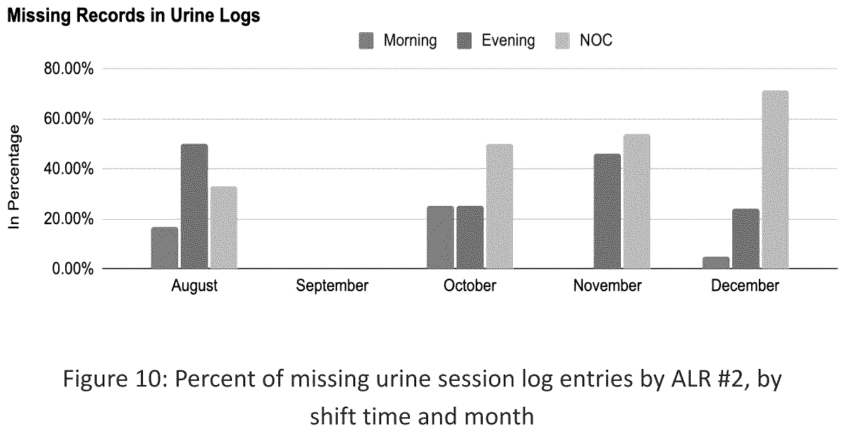

Analysis of the TL Sensor data to ALR #2 operator data showed that over the course of five months for the three residents analyzed, 53.05% of total stool sessions that were captured by the TL Sensor were missed by ALR #2 operators. Overall, 53% of stool sessions and 63% or urine sessions were missed by ALR #2 on an average monthly basis. Analysis shows that missing stool sessions occur on the same dates as missing urination sessions. This shows that ALR #2 care staff logged both stool and urination sessions likely at the same time, rather than updating them at the exact time of toileting. FIGS. 9 and 10 show missed entries for stool and urine sessions, respectively, by order of shift time (i.e., morning, evening, NOC), and month. From this, the less supported and typically less skilled overnight staff most often failed to enter any data.

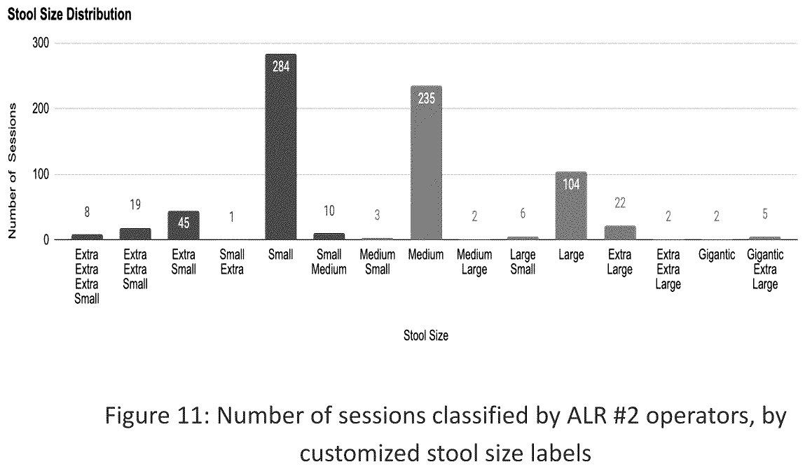

Although the logging format for ALR #2 appeared to be more complete than that of ALR #1, there seemed to be a great deal of subjectivity describing differences in stool consistency and size. There did not appear to be a robust set of instructions that described these differences in stool characteristics, but rather a vast range of descriptions, creating a great deal of nuance amongst operators. FIG. 11 demonstrates the distribution in stool size and the variability in their descriptors.

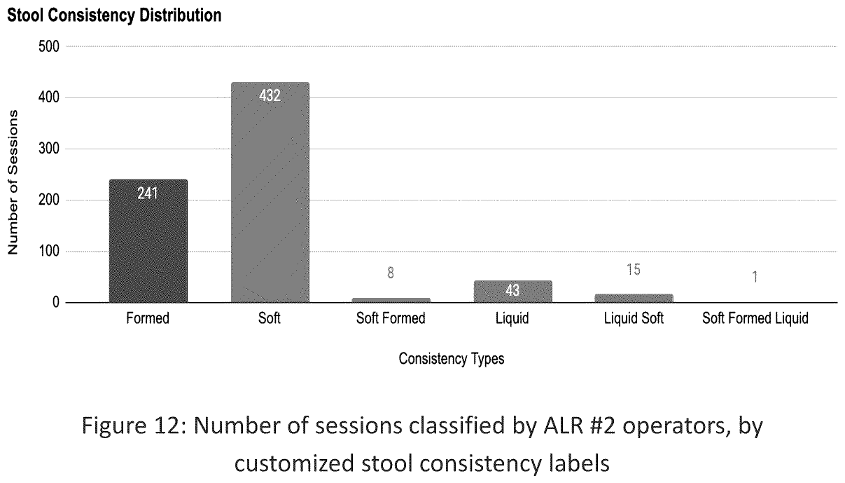

As seen in the results, the majority of stool sessions were categorized as small, medium, and large. However, there are several other categories that can create inconsistencies in ALR #2's stool size classification. FIG. 12 demonstrates the variability in stool consistency distribution descriptors.

As seen above, the majority of stool sessions were classified as formed, soft, or liquid, however there exists other categories, contributing to subjectivity and inconsistency in ALR #2's logging process. The TL Sensor's ability to classify stool consistency (i.e., formed and unformed) and size (i.e., small, medium, large) into distinct categories is intended to eliminate subjectivity and make it easier to identify clinically significant patterns in stool and urine changes when they occur (See Case studies Section).

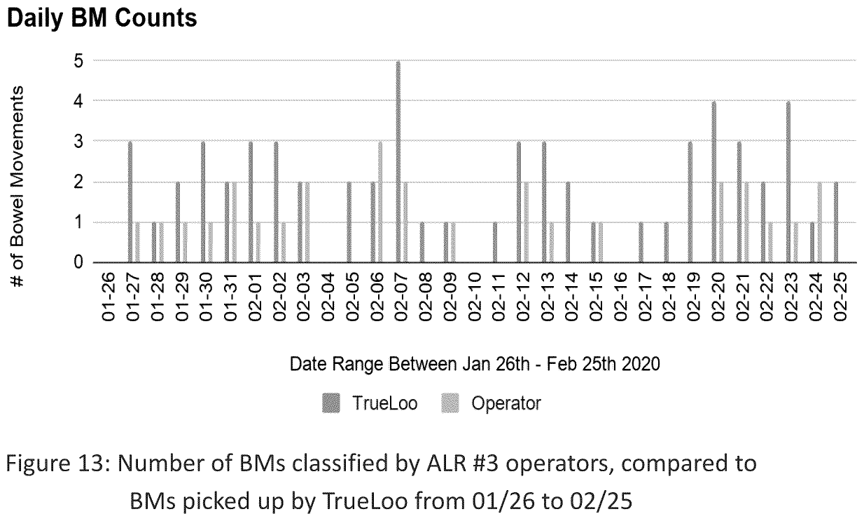

Analysis of ALR #3—Data was collected from a single resident at ALR #3. The operator logs for this community were maintained and updated electronically. The log entries were entered by 31 caregivers across the community from January 26th to Feb. 25, 2020. For simplicity, the analysis considered operator log outcomes such as “Self Toilets”, “None”, “Not recorded” as no stool activity was recorded. After comparing all of the log entries entered by the operator to the data picked up by the TL Sensor, it was determined that 54% of stool sessions were missed by the operator for this particular resident as can be seen in FIG. 13.

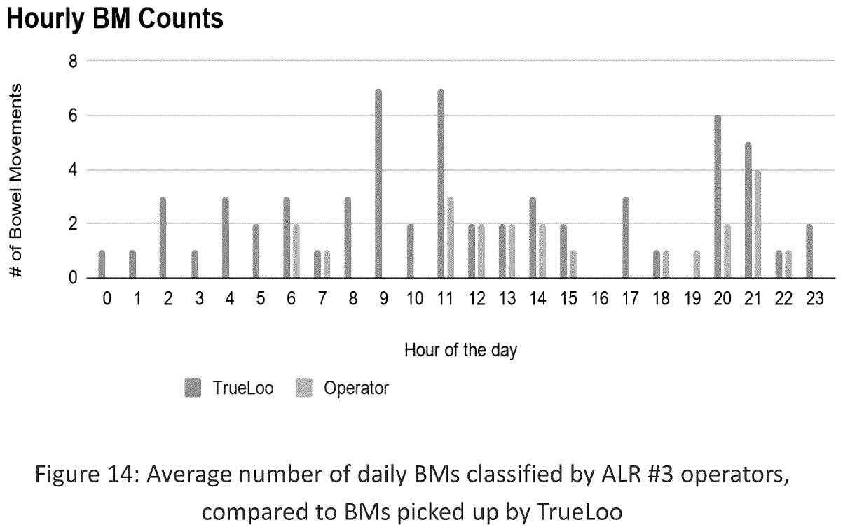

More specifically, the majority of stool sessions (approximately 81%) were missed during the overnight shift (11 PM to 6 AM). There are no records that reflect caregivers assisting residents with toileting from 11 PM to 5 AM; there are only “Self Toilets” sessions, which indicates that no stool sessions were directly logged by caregivers during this time period. The comparison between the number of bowel movements logged hourly on average by the operator and the TL Sensor can be seen in FIG. 14.

Besides operators missing more than half of bowel movement sessions for this resident, the analysis further determined that 100% of urine sessions and 100% of unformed stool sessions that were captured by the TL Sensor failed to be captured by operators. This is especially concerning as stool consistency and urination frequency have shown to be significant markers in recognizing stool and urine patterns.

### Case Studies

Comparisons between the TL Sensor data and clinical findings (i.e., clinically significant events that were self-reported by the participant or determined by reviewing the participant's medical records) were further investigated in order to corroborate three case studies that demonstrate the clinical significance of monitoring changes in stool and urine characteristics in high-acuity individuals. More specifically, these case studies provide evidence that the implementation of the TL Sensor system can enable non-clinical interventions, escalate care, and monitor chronic conditions in an older population.

Case Study #1: Recurrent Clostridium difficile (C. Diff)

An 89-year-old man living in an independent senior-living community experienced an unusual episode of dark, liquid stool on April 30. The resident visited the PCP on the same day and was prescribed 125 mg of vancomycin every 6 hours. Symptoms due to C. diff generally began resolving within 72 hrs of vancomycin treatment; however, diarrhea continued for 6 days through May 4th. The resident made a second visit to the PCP on May 5th, where labs were ordered and vancomycin was re-prescribed. The days following, persistent diarrhea and darker stool color improved though with intermittent instances of liquid diarrhea persisting until May 19th at which time repeat lab evaluation showed recurrent C. Diff infection. The resident was prescribed a stronger treatment, fidaxomicin, to improve symptoms. On June 2nd, the last day of the fidaxomicin treatment, the resident visited the PCP to follow up on treatment progress. No changes in care were made by the PCP and treatment was completed. On June 10th, severe diarrhea recurred. On June 14th, the resident experienced 10 sessions of liquid diarrhea in less than 24 hours. On June 15th, the resident was admitted to a skilled nursing facility due to frequent falls caused by dehydration due to diarrhea, as a result of recurrent C. Diff. The resident remained in the skilled nursing facility until July 7th.

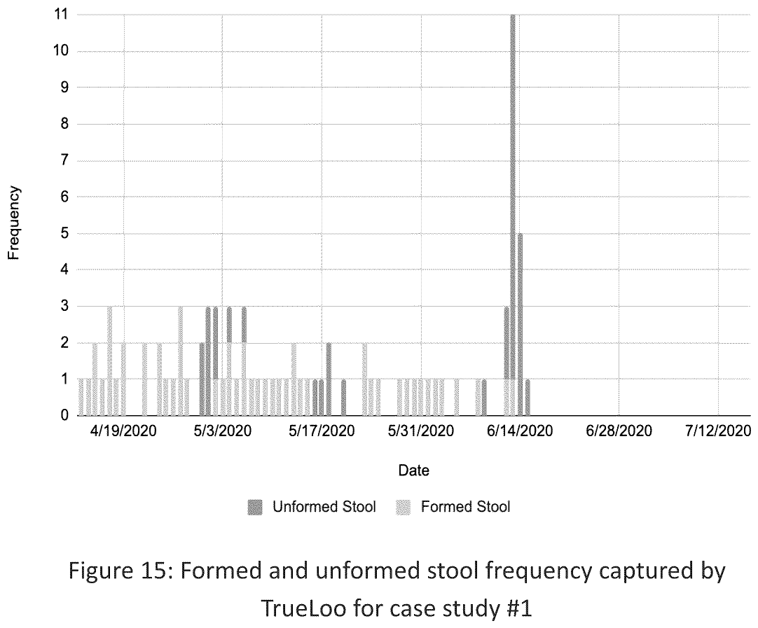

The analysis and aggregation of retrospective data captured by the TL Sensor demonstrates that the system established in advance that the initial treatment was ineffective. Furthermore, the TL Sensor recorded dramatic changes in the resident's stool habits days before falls had reportedly occurred. On May 4th, 72 hours after the initial treatment was prescribed, the TL Sensor captured that the resident's diarrhea symptoms persisted. Had the resident's primary care physician been informed of the inefficacy of the prescribed treatment, their treatment plan may have differed. Moreover, the TL Sensor's ability to monitor the persistent diarrhea episodes and detect a resurfacing on June 10th could have warned the resident to address their poor hydration status. In turn, this may have decreased the risk of falling and furthermore decreased the overall chances of admission to a skilled nursing facility. As supported by both the literature and the TL Sensor data, failure to pick up changes in stool and urine characteristics can lead to the exacerbation of clinical conditions such as C. Diff. The TL Sensor's ability to detect poor hydration status and monitor the efficacy of prescribed treatments by analyzing these changes can enable non-clinical interventions that can improve the resident's quality of life. FIG. 15 highlights the changes in stool frequency and consistency exhibited by this resident. As seen, the admission to skilled nursing immediately precedes an evident increase in both formed and unformed stool frequency.

### Case Study #2: Gastrointestinal Bleeding

A 91-year-old female on blood thinners had a change in stool color in late November that had gone unnoticed. Her stool became black and suspected frank blood was detected in her stool on multiple occasions in both early and late December. The resident was unaware of this change in their stool. She complained of unusual symptoms of weakness and dizziness in early January. Community staff called an ambulance and the resident was hospitalized on January 1st. Lab results showed that an upper GI bleed had occurred, resulting in overwhelming blood loss, dehydration, and acute kidney injury.

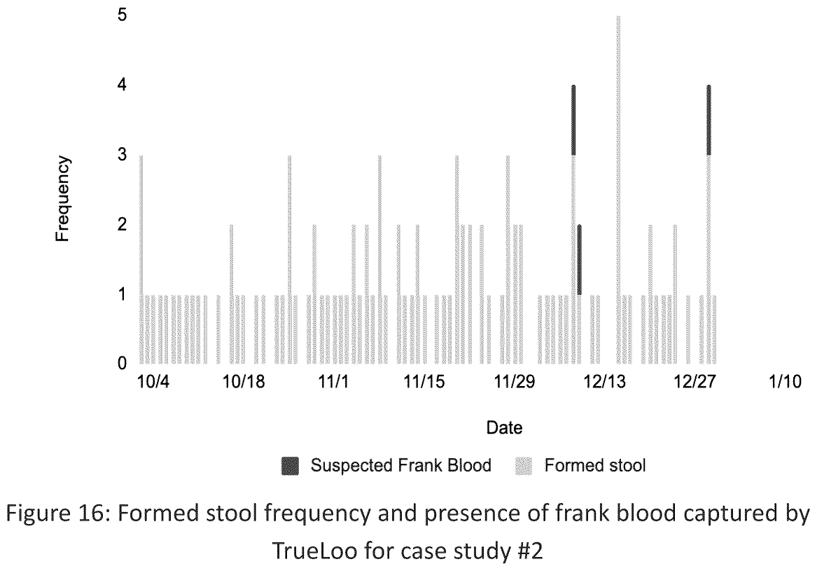

Retrospective data analysis from the TL Sensor demonstrated an unusual change in stool color on November 28th. Following this, suspected frank blood was imaged by the TL Sensor on two consecutive days, December 8th and 9th. Finally, the TL Sensor captured a more significant indication of frank blood on December 28th, three days prior to the resident being hospitalized. The resident was discharged from the hospital on February 12th. the TL Sensor ability to detect changes in stool characteristics (i.e., changes in stool color) shows that the system can provide early information to caregivers who can escalate care. All in all the TL Sensor data supported the diagnosis and demonstrated to have captured symptoms weeks prior to the resident being hospitalized. Since the resident was at a higher risk of gastrointestinal bleeding due to their anticoagulation, this key finding could have prompted the resident to seek treatment earlier, preventing the resident's condition from deteriorating. FIG. 16 highlights the suspected frank blood that was detected by the TL Sensor, along with the reported hospitalization, demonstrated by the absence of data from January 1st onwards.

### Case Study #3: Urinary Tract Infections and Falls

A 91-year-old female with a history of recurrent UTIs and resistance to antibiotics presented with cloudy urine and an increase in urine frequency on multiple occasions. The first time that the resident demonstrated dramatic changes in their urine habits is in late February and early March. After an abnormal urinalysis on March 3rd, the resident was diagnosed with a urinary tract infection and prescribed Bactrim, given their antibiotic resistance to other antibiotics like Ciprofloxacin. Following about three days of treatment, the resident experienced a decrease in urination frequency and stabilizes, with virtually no instances of cloudy urine.

A few months later, in early May, the resident experienced a change in urination frequency and clarity. The resident was unable to get a urinalysis done due to COVID restrictions. They are prescribed antibiotics in mid-May with a suspected UTI diagnosis by their PCP. Three days following the prescribed treatment, the resident experienced a fall due to suspected dehydration caused by the diagnosed UTI.

Three days following the prescribed treatment, the resident experienced a fall, which may have been associated with recurrent UTI given the association between UTI and an increased risk of fall. With UTIs accounting for over a third of all nursing home-associated infections, monitoring them is key to reducing the risk of exacerbated conditions (i.e., falls, kidney stones, chronic kidney failure, etc.)

The TL Sensor data detected a change in the resident's urination frequency in early May, well before the resident contacted their primary care physician for a video appointment and sought treatment. In a post-COVID world, where home care is highly preferred, the TL Sensor can relay information to caregivers that can make a lasting impact on those with chronic conditions. Information collected by the TL Sensor could have informed caregivers of the resident's change in urine habits, and could have significantly improved the resident's hydration status at an earlier point, decreasing the risk dehydration and associated falls.

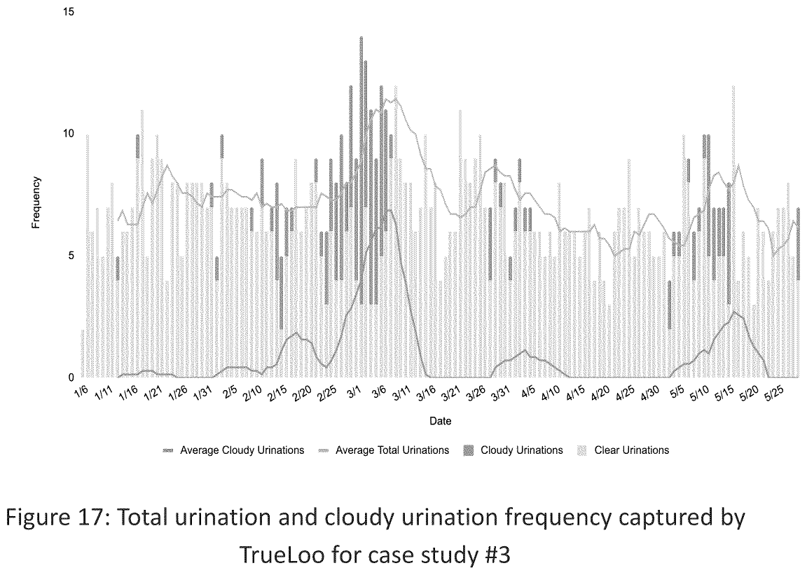

FIG. 17 highlights an evident increase in urination frequency exhibited by the resident. As seen, the dramatic increase in urination frequency correlates with the time of diagnosis. Furthermore, the spike in urination frequency on May 16th directly correlates with the date at which the resident experienced a fall, likely contributed by the diagnosed UTI and associated dehydration.

The study successfully reached the desired end points of creating a complete stool and urination dataset and comparing to traditional methods of data collection. The feasibility of the TL Sensor system to monitor and record clinically meaningful changes in stool and urine characteristics was demonstrated in three example case studies. Analysis of all monthly adverse episodes across the 98 participants, which includes residents in both independent and assisted living communities, demonstrated that 46.9% of the participants experienced a clinically significant event throughout the course of the clinical study. Furthermore, the study found that staff missed 50% of stool sessions, and residents failed to self-report 50% of adverse episodes, which validates the study's initial hypothesis regarding the unreliability and inaccuracy of these methods. These discrepancies in data collection clearly demonstrate the importance of the TL Sensor in enabling actionable clinical recommendations based on observable changes in excreta patterns. Finally, a high degree of user satisfaction was found in the exit survey, where the following data was collected:
95% of participants believe that the TL Sensor has the potential to help older adults.
91% of participants reported that using the TL Sensor required no effort.
73% of participants preferred the TL Sensor to send urgent alerts about their stool or urine to their caregiver.

The following exemplary and non-limiting embodiments are provided to further illustrate the disclosed principles. These examples are non-limiting and different implementations can be made without departing from the disclosed principles.

Example 1 is directed to a system to detect one or more health abnormality from a excrete of a patient, the system comprising: a memory circuitry; a processing circuitry in communication with the memory circuitry, the processing circuitry configured to: receive a training dataset having a plurality of data fields, each data field having a plurality of records; identify a first threshold from the plurality of records, the first threshold indicating presence of a first health abnormality when the first threshold is met or exceeded; receive a first measurement obtained from the excrete of the patient; correlate the first measurement with the first statistical threshold to identify presence of the first health abnormality; and communicate the identified first health abnormality.

Example 2 is directed to the system of Example 1, wherein the first measurement is determined from a plurality of records.

Example 3 is directed to the system of Example 1, wherein the first measurement is determined from a plurality of records and wherein at least two of the plurality of records are associated with different data fields.

Example 4 is directed to the system of Example 1, wherein the first health abnormality defines one or more of urinary tract infection, constipation, diarrhea and cloudy urine and internal bleeding.

Example 5 is directed to the system of Example 1, wherein the processor is further configured to identify a second threshold from the plurality of records, the second threshold indicating presence of a second health abnormality when the second threshold is met or exceeded.

Example 6 is directed to the system of Example 5, wherein the processor is further configured to identify a second threshold from the plurality of records, the second threshold indicating presence of a second health abnormality when one ore more of the first or the second threshold is met or exceeded.

Example 7 is directed to the system of Example 1, wherein the plurality of data fields are selected from the group consisting of: stool pH, color, volume and frequency.

Example 8 is directed to the system of Example 1, wherein the plurality of records define substantially similar measurements made at different times.

Example 9 is directed to the system of Example 1, wherein the steps of receiving the training dataset and identifying the first threshold define machine learning and wherein the steps of receiving the first measurement and correlating the first measurement define testing.

Example 10 is directed to a method to train an artificial intelligence (AI) to detect presence of a first health abnormality in a patient excrete, the method comprising: receiving a training dataset having a plurality of data fields, each data field having a plurality of records; identifying a first threshold from the plurality of records, the first threshold indicating presence of a first health abnormality when the first threshold is met or exceeded; receiving a first measurement obtained from the patient excrete; correlating the first measurement with the first statistical threshold to identify presence of the first health abnormality; and communicating the identified first health abnormality.

Example 11 is directed to the method of Example 10, wherein the first measurement is determined from a plurality of records associated with the excrete of the patient.

Example 12 is directed to the method of Example 10, wherein the first measurement is determined from a plurality of records and wherein at least two of the plurality of records are associated with different data fields.

Example 13 is directed to the method of Example 10, wherein the first health abnormality defines one or more of urinary tract infection, constipation, diarrhea and cloudy urine and internal bleeding.

Example 14 is directed to the method of Example 10, further comprising identifying a second threshold from the plurality of records, the second threshold indicating presence of a second health abnormality when the second threshold is met or exceeded.

Example 15 is directed to the method of Example 14, further comprising identifying a second threshold from the plurality of records, the second threshold indicating presence of a second health abnormality when one ore more of the first or the second threshold is met or exceeded.

Example 16 is directed to the method of Example 10, wherein the plurality of data fields are selected from the group consisting of: stool pH, color, volume and frequency.

Example 17 is directed to the method of Example 10, wherein the plurality of records define substantially similar measurements made at different times.

Example 18 is directed to the method of Example 10, wherein the steps of receiving the training dataset and identifying the first threshold define machine learning and wherein the steps of receiving the first measurement and correlating the first measurement define testing.

Example 19 is directed to a method to training an artificial intelligence (AI) to detect presence of a first health abnormality in an excrete of a patient, the method comprising: receiving multiple frames associated a patient excrete; labeling each of the multiple frames with an identifying label; deducing a session label based on the multiple frame labels; comparing the session labels with one or more threshold labels, each threshold label indicating presence of a health abnormality when the threshold is met or exceeded; and identifying presence of a first health abnormality when the threshold is exceeded.

Example 20 is directed to the method of Example 19, wherein the multiple frames define optical images taken during excretion of the patient.

Example 21 is directed to the method of Example 19, wherein the step of labeling each of the multiple frames further comprises measuring at least one physical attribute of the patent excrete.

Example 22 is directed to the method of Example 19, wherein the health abnormality defines one or more of urinary tract infection, constipation, diarrhea and cloudy urine and internal bleeding.

Example 23 is directed to the method of Example 19, further comprising identifying a second threshold from the plurality of records, the second threshold indicating presence of a second health abnormality when the second threshold is met or exceeded.

Example 24 is directed to the method of Example 23, further comprising identifying a second threshold from the plurality of records, the second threshold indicating presence of a second health abnormality when one ore more of the first or the second threshold is met or exceeded.

Example 25 is directed to the method of Example 21, wherein the physical attribute is selected from the group consisting of: stool pH, color, volume and frequency.

Example 26 is directed to a non-transitory computer-readable medium comprising a processor circuitry and a memory circuitry in communication with the processor circuitry and including instructions to provide multifactor authentication, the memory circuitry further comprising instructions to cause the processor to: receive multiple frames associated a patient excrete; label each of the multiple frames with an identifying label; deduce a session label based on the multiple frame labels; compare the session labels with one or more threshold labels, each threshold label indicating presence of a health abnormality when the threshold is met or exceeded; and identify presence of a first health abnormality when the threshold is exceeded.

Example 27 is directed to the medium of Example 26, wherein the multiple frames define optical images taken during excretion of the patient.

Example 28 is directed to the medium of Example 26, wherein the instructions further cause the processor to label each of the multiple frames by measuring at least one physical attribute of the excrete.

Example 29 is directed to the medium of Example 26, wherein the health abnormality defines one or more of urinary tract infection, constipation, diarrhea and cloudy urine and internal bleeding.

Example 30 is directed to the medium of Example 26, wherein the instructions further cause the processor to identify a second threshold from the plurality of records, the second threshold indicating presence of a second health abnormality when the second threshold is met or exceeded.

Example 31 is directed to the medium of Example 30, wherein the instructions further cause the processor to identify a second threshold from the plurality of records, the second threshold indicating presence of a second health abnormality when one ore more of the first or the second threshold is met or exceeded.

Example 32 is directed to the medium of Example 31, wherein the physical attribute is selected from the group consisting of: stool pH, color, volume and frequency.

The embodiments presented herein are merely illustrative of certain principles of the disclosure.

APPENDIX A
Monthly Nurse Assessment Questionnaire to independent residents

How are you feeling today?
Enter resident's name:
Since the last assessment have you visited the doctor or hospital/ER for a problem
related to your stool or urine?
If yes,
Which hospital did you visit and when?
What was the reason you visited the doctor or hospital?
How long were you in the hospital or times visiting the doctor?
What symptoms or signs did you have leading up to visiting the doctor or hospital?
Did you receive a prescription or follow up appointment as a result of visiting the
doctor or hospital?
If yes to previous question enter information on medication or follow up here:
When is your next visit with your primary care physician/doctor?
Are there any changes in your medications since the last assessment?
Have you been prescribed antibiotics since the last assessment?
Have you had any problems with dehydration, or a UTI since the last assessment?
Have you observed cloudy or abnormally dark urine since the last assessment?
Have you experienced diarrhea since the last assessment?
Have you experienced constipation since the last assessment?
Have you observed blood in the toilet since the last assessment?
Has there been a major change in your diet since the last assessment?
Since the last assessment have you added fiber or a stool softener?
Have you changed your fluid intake since the last time we spoke with you?
Have you noticed any changes in your bowel movements since the last time we
spoke with you?
Have you noticed any changes in your urinations since the last time we spoke with
you?
Any other changes you want to let me know about?
Would you be willing to fill out a release so we might access records?

APPENDIX B
Exit survey on User Acceptance and Satisfaction

Name of Participant
Name of Community
How long did the resident use the new toilet seat?
Regarding just your experience with the new toilet seat, how much effort was
required to use it?
How did the new toilet seat compare to your previous toilet seat?
What do you think of the potential of this toilet monitoring system to help older
adults?
If the system provided you alerts for potential health issues would you find that
valuable?
If you weren't feeling well and needed to visit the doctor, would you share
information from the toilet seat to help in the diagnosis?
Would you feel more comfortable if we sent urgent alerts about your stool or urine
to your caregivers instead of you having to tell them?
What did you like most about the new toilet seat?
What do you think can be improved in the toilet seat?
Name of Primary Physician
Name of Medical Group

What is claimed is:

1. A system to detect one or more health abnormality from a excrete of a patient, the system comprising:
a memory circuitry;
a processing circuitry in communication with the memory circuitry, the processing circuitry configured to:
receive a training dataset having a plurality of data fields, each data field having a plurality of records;
identify a first threshold from the plurality of records, the first threshold indicating presence of a first health abnormality when the first threshold is met or exceeded;
receive a first measurement obtained from the excrete of the patient;
correlate the first measurement with the first statistical threshold to identify presence of the first health abnormality; and
communicate the identified first health abnormality.

2. The system of claim 1, wherein the first measurement is determined from a plurality of records.

3. The system of claim 1, wherein the first measurement is determined from a plurality of records and wherein at least two of the plurality of records are associated with different data fields.

4. The system of claim 1, wherein the first health abnormality defines one or more of urinary tract infection, constipation, diarrhea and cloudy urine and internal bleeding.

5. The system of claim 1, wherein the processor is further configured to identify a second threshold from the plurality of records, the second threshold indicating presence of a second health abnormality when the second threshold is met or exceeded.

6. The system of claim 5, wherein the processor is further configured to identify a second threshold from the plurality of records, the second threshold indicating presence of a second health abnormality when one ore more of the first or the second threshold is met or exceeded.

7. The system of claim 1, wherein the plurality of data fields are selected from the group consisting of: stool pH, color, volume and frequency.

8. The system of claim 1, wherein the plurality of records define substantially similar measurements made at different times.

9. The system of claim 1, wherein the steps of receiving the training dataset and identifying the first threshold define machine learning and wherein the steps of receiving the first measurement and correlating the first measurement define testing.

10. A method to train an artificial intelligence (AI) to detect presence of a first health abnormality in a patient excrete, the method comprising:
receiving a training dataset having a plurality of data fields, each data field having a plurality of records;
identifying a first threshold from the plurality of records, the first threshold indicating presence of a first health abnormality when the first threshold is met or exceeded;
receiving a first measurement obtained from the patient excrete;
correlating the first measurement with the first statistical threshold to identify presence of the first health abnormality; and
communicating the identified first health abnormality.

11. The method of claim 10, wherein the first measurement is determined from a plurality of records associated with the excrete of the patient.

12. The method of claim 10, wherein the first measurement is determined from a plurality of records and wherein at least two of the plurality of records are associated with different data fields.

13. The method of claim 10, wherein the first health abnormality defines one or more of urinary tract infection, constipation, diarrhea and cloudy urine and internal bleeding.

14. The method of claim 10, further comprising identifying a second threshold from the plurality of records, the second threshold indicating presence of a second health abnormality when the second threshold is met or exceeded.

15. The method of claim 14, further comprising identifying a second threshold from the plurality of records, the second threshold indicating presence of a second health abnormality when one ore more of the first or the second threshold is met or exceeded.

16. The method of claim 10, wherein the plurality of data fields are selected from the group consisting of: stool pH, color, volume and frequency.

17. The method of claim 10, wherein the plurality of records define substantially similar measurements made at different times.

18. The method of claim 10, wherein the steps of receiving the training dataset and identifying the first threshold define machine learning and wherein the steps of receiving the first measurement and correlating the first measurement define testing.

19. A method to training an artificial intelligence (AI) to detect presence of a first health abnormality in an excrete of a patient, the method comprising:
receiving multiple frames associated a patient excrete;
labeling each of the multiple frames with an identifying label;
deducing a session label based on the multiple frame labels;
comparing the session labels with one or more threshold labels, each threshold label indicating presence of a health abnormality when the threshold is met or exceeded; and
identifying presence of a first health abnormality when the threshold is exceeded.

20. The method of claim 19, wherein the multiple frames define optical images taken during excretion of the patient.

21. The method of claim 19, wherein the step of labeling each of the multiple frames further comprises measuring at least one physical attribute of the patent excrete.

22. The method of claim 19, wherein the health abnormality defines one or more of urinary tract infection, constipation, diarrhea and cloudy urine and internal bleeding.

23. The method of claim 19, further comprising identifying a second threshold from the plurality of records, the second threshold indicating presence of a second health abnormality when the second threshold is met or exceeded.

24. The method of claim 23, further comprising identifying a second threshold from the plurality of records, the second threshold indicating presence of a second health abnormality when one ore more of the first or the second threshold is met or exceeded.

25. The method of claim 21, wherein the physical attribute is selected from the group consisting of: stool pH, color, volume and frequency.

26. A non-transitory computer-readable medium comprising a processor circuitry and a memory circuitry in communication with the processor circuitry and including instructions to provide multifactor authentication, the memory circuitry further comprising instructions to cause the processor to:
receive multiple frames associated a patient excrete;
label each of the multiple frames with an identifying label;
deduce a session label based on the multiple frame labels;
compare the session labels with one or more threshold labels, each threshold label indicating presence of a health abnormality when the threshold is met or exceeded; and
identify presence of a first health abnormality when the threshold is exceeded.

27. The medium of claim 26, wherein the multiple frames define optical images taken during excretion of the patient.

28. The medium of claim 26, wherein the instructions further cause the processor to label each of the multiple frames by measuring at least one physical attribute of the excrete.

29. The medium of claim 26, wherein the health abnormality defines one or more of urinary tract infection, constipation, diarrhea and cloudy urine and internal bleeding.

30. The medium of claim 26, wherein the instructions further cause the processor to identify a second threshold from the plurality of records, the second threshold indicating presence of a second health abnormality when the second threshold is met or exceeded.

31. The medium of claim 30, wherein the instructions further cause the processor to identify a second threshold from the plurality of records, the second threshold indicating presence of a second health abnormality when one ore more of the first or the second threshold is met or exceeded.

32. The medium of claim 31, wherein the physical attribute is selected from the group consisting of: stool pH, color, volume and frequency.
